(19) United States
(12) Patent Application Publication
(10) Pub. No.: US 2009/0042781 A1
(43) Pub. Date: Feb. 12, 2009

(54) Methods for Treating Diabetes

(75) Inventors: Jacob Sten Petersen, Kobenhavn O (DK); Lars Hansen, Frederiksberg (DK); Elisabeth D. Galsgaard, Naerum (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordisk A/S, Bagsvaerd (DK)

(21) Appl. No.: 11/630,068

(22) PCT Filed: Jun. 23, 2005
(86) PCT No.: PCT/EP05/52931

§ 371 Date: Jan. 9, 2008

(30) Foreign Application Priority Data
Jun. 28, 2004 (DK) PA 2004 01010

(51) Int. Cl. A61K 38/22; A61K 31/435; A61K 31/192

(52) U.S. Cl. 514/12; 514/338; 514/571

(57) ABSTRACT

Methods for treating diabetes by increasing the insulin secretion by administration of a GLP-1 receptor agonist and/or a DPP-IV inhibitor in combination with a proton pump inhibitor and optionally a PPAR agonist are provided.

### FIELD OF THE INVENTION

The present invention relates to methods and compositions useful in the treatment of glycometabolic disorders.

### BACKGROUND OF THE INVENTION

Diabetes mellitus comprises a group of diseases that result in elevation of the blood glucose level because of relative or absolute deficiency in the pancreatic hormone insulin. Insulin is secreted from the pancreas into the blood in response to the blood glucose level, and a major function is to direct blood glucose into body stores, whereby the blood glucose level is controlled.

Chronic elevation of the blood glucose level is the most obvious metabolic effect in diabetes and is associated with progressive damage to blood vessels. This may lead to heart attack, stroke, blindness, peripheral nerve dysfunction, and kidney failure. Diabetes is known in the Type I form and in the Type II form. Type I diabetes is related to an immunological destruction of the insulin secreting pancreatic β-cells. Type II diabetes it is related to a combination of β-cell deficiency and peripheral insulin resistance. Type II diabetes is a slowly progressive disease, and β-cell function continues to deteriorate despite any of the currently available treatments. Diabetes is a major public health-problem affecting at least 5 million and probably as many as 10 million Americans.

Currently, Type I and late stage Type II diabetes are treated by administration of insulin or insulin compounds to the patients. Early stage type II diabetes is normally treated with oral drugs which increase insulin secretion from the pancreas or which increase tissue sensitivity towards insulin. Unfortunately, neither insulin replacement therapy or the above mentioned oral drugs restore normoglycemia, and postprandial blood glucose levels are typically excessively high, which in many cases ultimately leads to the above mentioned diabetic complications. Thus, there are obvious advantages if an efficacious treatment could be developed which re-establishes the ability of the pancreas to produce insulin in response to the blood glucose level.

Transplantation of β-cells has been suggested, however, transplantations require finding a suitable donor, surgical procedures, and graft acceptance.

WO 00/07617 discloses that GLP-1 and analogues thereof increase the number and size of β-cells.

WO 00/09666 discloses that GLP-1 and growth factors with substantially homologous amino acid sequences are capable of inducing differentiation of non-insulin dependent cells into insulin producing cells.

WO 01/39784 discloses a method for treating patients with diabetes mellitus, the method comprising isolating stem cells from a pancreas, treating said stem cells ex vivo with e.g. certain specified GLP-1 receptor agonists to provide progenitor cells, which upon trans-plantation into the patients differentiate into insulin producing D-cells.

WO 95/19785 discloses a method for treating diabetes mellitus, the method comprising administration of a gastrin receptor ligand, such as gastrin itself, together with an endothelial growth factor (EGF) receptor ligand, such as EGF itself. In a particular embodiment, the method comprises the administration of a compound which induces gastrin production in the body and an EGF receptor ligand. One such gastrin inducer is Omeprazole.

WO 04/037195 discloses that GLP-1 receptor ligands in combination with gastrin may be used to treat diabetes mellitus.

Hammer et al in Scan. J. Gastroenterology, 33, 595-599, 1998 disclose that omeprazole, ciprofibrate and the combination of omeprazole and ciprofibrate when administered by gastric gavage give rise to an increase in the serum gastrin level.

Dipeptidyl peptidase-IV (DPP-IV), a serine protease belonging to the group of postproline/alanine cleaving amino-dipeptidases, specifically removes the two N-terminal amino acids from proteins having proline or alanine in position 2.

DPP-IV has been implicated in the control of glucose metabolism because its sub-strates include the insulinotropic hormones Glucagon like peptide-1 (GLP-1) and Gastric inhibitory peptide (GIP). GLP-1 and GIP are active only in their intact forms, removal of their two N-terminal amino acids inactivates them.

In vivo administration of synthetic inhibitors of DPP-IV prevents N-terminal degradation of GLP-1 and GIP, resulting in higher plasma concentrations of these hormones.

Inhibitors of DPP-IV have previously been disclosed in WO 95/15309 (Ferring B. V.), WO 98/19998, WO 00/34241, U.S. Pat. No. 6,124,305 (Novartis A G), WO 03/00180 (Merck & Co.), and WO 02/38541 (Taisho Pharmaceutical Co.).

### SUMMARY OF THE INVENTION

In an embodiment, the invention relates to methods of
increasing,
preserving,
or reducing the rate of loss,
   in insulin secretion in a patient, the method comprising the administration of therapeutically effective amounts of a GLP-1 receptor agonist and/or a DPP-IV inhibitor, in combination with a proton pump inhibitor and optionally a PPAR agonist to a patient in need thereof.

In an embodiment, the invention relates to methods of increasing, preserving or reducing the rate of loss of β-cell function in a patient, the method comprising the administration of therapeutically effective amounts of a GLP-1 receptor agonist and/or a DPP-IV inhibitor, in combination with a proton pump inhibitor and optionally a PPAR agonist to a patient in need thereof.

In an embodiment, the invention relates to methods of increasing, preserving or reducing the rate of loss in the number and/or size of β-cells in a patient, the method comprising the administration of therapeutically effective amounts of a GLP-1 receptor agonist and/or a DPP-IV inhibitor, in combination with a proton pump inhibitor and/or a PPAR agonist to a patient in need thereof.

In an embodiment, the invention relates to the treatment of diseases benefiting from an increase, preservation or reduction in rate of loss in the insulin secretion, the method comprising the administration of therapeutically effective amounts of a GLP-1 receptor agonist and/or a DPP-IV inhibitor, in combination with a proton pump inhibitor and optionally a PPAR agonist to a patient in need thereof.

In an embodiment, the invention relates to the treatment of diseases benefiting from an increase, preservation or reduction in rate of loss in the β-cell function, the method comprising the administration of therapeutically effective amounts of a GLP-1 receptor agonist and/or a DPP-IV inhibitor in combination with a proton pump inhibitor and optionally a PPAR agonist to a patient in need thereof.

In an embodiment, the invention relates to the treatment of diseases benefiting from an increase, preservation or reduction in rate of loss of the number and/or size of the β-cells, the method comprising the administration of therapeutically effective amounts of a GLP-1 receptor agonist and/or a DPP-IV inhibitor, in combination with a proton pump inhibitor and optionally a PPAR agonist to a patient in need thereof.

In an embodiment, the invention relates to the use of a GLP-1 receptor agonist and/or a DPP-IV inhibitor, in combination with a proton pump inhibitor and optionally a PPAR agonist in the manufacture of a medicament for the increase, preservation or reduction in rate of loss of the insulin secretion in a subject.

In an embodiment, the invention relates to the use of a GLP-1 receptor agonist and/or a DPP-IV inhibitor, in combination with a proton pump inhibitor and optionally a PPAR agonist in the manufacture of a medicament for the increase, preservation or reduction of rate of loss of the β-cell function of a subject.

In an embodiment, the invention relates to the use of a GLP-1 receptor agonist and/or a DPP-IV inhibitor, in combination with a proton pump inhibitor and optionally a PPAR agonist in the manufacture of a medicament for the increase, preservation or reduction of loss in the number and/or size of β-cells in a subject.

In an embodiment, the invention relates to a pharmaceutical composition comprising a GLP-1 receptor agonist and/or a DPP-IV inhibitor, in combination with a proton pump inhibitor and optionally a PPAR agonist.

In an embodiment, the invention relates to a kit comprising a container with a GLP-1 receptor agonist and/or a DPP-IV inhibitor, and a container with a proton pump inhibitor and optionally a container with a PPAR agonist.

In an embodiment, the invention relates to a method of promoting the sales of a composition or kit comprising a GLP-1 receptor agonist and/or a DPP-IV inhibitor, a proton pump inhibitor, and optionally a PPAR agonist, said method comprising the public distribution of the information that administration of said composition or kit is associated with β-cell proliferation and/or β-cell neogenesis.

### DEFINITIONS

In the present context, the word “a” means one or more.

In the present context, a “GLP-1 receptor agonist” is taken to be any compound, including peptides and non-peptide compounds, which fully or partially activates the human GLP-1 receptor. In a preferred embodiment, the “GLP-1 receptor agonist” is any peptide or non-peptide molecule that binds to a GLP-1 receptor with an affinity constant (K_{D}) or a potency (EC₅₀) below 1 μM, such as below 100 nM as measured by methods known in the art (see WO 98/08871, which is incorporated herein in its entirety) and exhibits insulinotropic activity, where insulinotropic activity may be measured using in vivo or in vitro assays known to those skilled in the art. Particular examples of GLP-1 receptor agonists include human GLP-1 and GLP-1 compounds. Human GLP-1 is a 37 amino acid residue peptide originating from preproglucagon which is synthesised i.a. in the L-cells in the distal ileum, in the pancreas and in the brain. GLP-1 is an important gut hormone with regulatory function in glucose metabolism and gastrointestinal secretion and metabolism. Processing of preproglucagon to give GLP-1(7-36)-amide, GLP-1(7-37) and GLP-2 occurs mainly in the L-cells. The fragments GLP-1(7-36)-amide and GLP-1(7-37) are both glucose-dependent insulinotropic agents. In the past decades a number of structural analogues of GLP-1 have been isolated from the venom of the Gila monster lizards (Heloderma suspectum and Heloderma horridum). Exendin-4 is a 39 amino acid residue peptide isolated from the venom of Heloderma horridum, and this peptide shares 52% homology with GLP-1. Exendin-4 is a potent GLP-1 receptor agonist which has been shown to stimulate insulin release and ensuring lowering of the blood glucose level when injected into dogs. The group of human GLP-1(1-37) and exendin-4(1-39) and insolinotropic fragments, analogues and derivatives thereof (designated GLP-1 compounds herein) are all applicable in the present invention. Insulinotropic fragments of GLP-1(1-37) are insulinotropic peptides for which the entire sequence can be found in the sequence of GLP-1(1-37) and where at least one terminal amino acid has been deleted. Insulinotropic analogs of GLP-1(1-37) and exendin-4(1-39) refer to the respective molecules wherein one or more of the amino acids residues have been exchanged with other amino acid residues and/or from which one or more amino acid residues have been deleted and/or from which one or more amino acid residues have been added with the proviso that said analogue either is insulinotropic or is a prodrug of an insulinotropic compound. Insulinotropic derivatives of GLP-1(1-37), exendin-4(1-39) and analogs thereof are what the person skilled in the art considers to be derivatives of these peptides, i.e. having at least one sub-stituent which is not present in the parent peptide molecule with the proviso that said derivative either is insulinotropic or is a prodrug of an insulinotropic compound. Examples of sub-stituents are amides, carbohydrates, alkyl groups PEG, and lipophilic substituents. Derivatives of the GLP-1 receptor agonists may be long-acting (protracted). A long-acting derivatives has a longer plasma half-life as compared to the parent peptide. Examples of GLP-1 compounds are described in, e.g. WO 98/08871, WO 99/43706, U.S. Pat. No. 5,424,286 and WO 00/09666, which are all enclosed herein in their entirety.

In the present context, a “DPP-IV inhibitor” refers to DPP-IV which as used herein is intended to mean Dipeptidyl peptidase IV (EC 3.4. 14.5; DPP-IV), also known as CD26. DPP-IV cleaves a dipeptide from the N terminus of a polypeptide chain containing a proline or alanine residue in the penultimate position. An inhibitor of DPP-IV is a compound which lowers the activity or efficacy of DPP-IV.

In the present context, a “proton pump inhibitor” is intended to indicate a compound which inhibits the hydrogen-potassium adenosine triphosphate enzyme system of the gastric parietal cells, whereby gastric acid secretion from these cells is prevented. Proton pump inhibitors are used in the treatment of e.g. gastric ulcers, and prominent examples of proton pump inhibitor drugs are omeprazole, esomeprazole, iansoprazole, pantoprazole and rabeprazole.

“Peroxisome proliferators-activated receptors” (PPAR) are members of the nuclear hormone receptor superfamily, and they are activated, e.g. by saturated and unsaturated fatty acids and various synthetic ligands. PPAR are heterogeneous, and three sub-types have been isolated to date, namely PPARα, PPARδ and PPARγ. Compounds which are agonist of PPARα and/or PPARδ and/or PPARγ are regarded as PPAR agonists

PPARα is mostly expressed in tissue with a high rate of fatty acid catabolism, such as the liver, and it is generally involved in lipid metabolism. A PPARα agonist is a compound which activates the PPARα receptor, and such compounds can be identified using a PPARα transactivation assay as disclosed in WO 02/28821, Beispiel A, which is incorporated herein in its entirety. Any compound with an EC₅₀ below 20 uM is regarded as a PPARα agonist. Fibrates are particular examples of PPARα agonists.

Activation of PPARδ has been shown to lead to increased levels of HDL cholesterol in dbldb mice. Further, a PPARδ agonist when dosed to insulin-resistant middle-aged obese rhesus monkeys caused a dramatic dose-dependent rise in serum HDL cholesterol while lowering the levels of LDL cholesterol, fasting triglycerides and fasting insulin. A PPARδ agonist is a compound which activates the PPARδ receptor, and such compounds can be identified using a PPARδ transactivation assay as disclosed in WO 04/037776, which is incorporated herein in its entirety. Any compound with an EC₅₀ below 20 uM is regarded as a PPARδ agonist.

PPARγ, is mostly present in tissue with metabolic significance, e.g. adipose tissue, skeletal muscles and in the liver A PPARγ agonist is a compound which activates the PPARγ receptor, and such compounds can be identified using a PPARγ transactivation assay as disclosed in e.g. Sauerberg et al. J. Med. Chem. 2002, 45, 789-804, which is incorporated herein in its entirety. Any compound with an EC₅₀ below 20 uM is regarded as a PPARγ agonist A particular type of PPARγ agonists is thiazolidine compounds (TZD) which are characterised by the presence of the thiazolidine-2,4-dione moiety

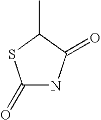

in the molecular structure. Relevant TZD include e.g. balaglitazone, troglitazone, ciglitazone, pioglitazone, rosiglitazone, isaglitazone, darglitazone, englitazone, CS-011/CI-1037 or T174 or the compounds disclosed in WO 97/41097, WO 97/41119, WO 97/41120, WO 00/41121 and WO 98/45292, which are all incorporated herein by reference.

There are also non-thiazolidine PPARγ agonist, such as GI 262570, YM-440, MCC-555, JTT-501, AR-H039242, KRP-297, GW-409544, CRE-16336, AR-H049020, LY510929, MBX-102, CLX-0940, GW-501516 or the compounds disclosed in WO 99/19313 (e.g. Ragaglitazar), WO 00/50414, WO 00/63191, WO 00/63192, WO 00/63193 (Dr. Reddy's Research Foundation) and WO 00/23425, WO 00/23415, WO 00/23451, WO 00/23445, WO 00/23417, WO 00/23416, WO 00/63153, WO 00/63196, WO 00/63209, WO 00/63190 and WO 00/63189 (Novo Nordisk A/S), which are incorporated herein by reference.

Certain compounds are dual- or triple-acting PPAR agonist, i.e. they are agonist of PPARα and PPARδ, agonists of PPARα and PPARγ, agonists of PPARδ and PPARγ or agonist of PPARα, PPARδ and PPARγ. Dual- and triple acting PPAR agonists are useful in the methods, uses, compositions and kits of the present invention.

DPP-IV inhibitors are compounds such as vildagliptin, MK-0431, BMS-477118 (saxagliptin), PSN-9301, 823093, SYR-322, SYR-619, 815541, 825964, TA-6666 or TS-021;

In the present context, a “medicament” is intended to include one composition comprising all the therapeutically active agents to be used in the methods of the present invention, and also to include kits comprising two or more containers which in combination comprise all the therapeutically active agents to be used in the methods of the present invention.

A “therapeutically effective amount” of a compound as used herein means an amount sufficient to cure, alleviate or partially arrest the clinical manifestations of a given disease and its complications. An amount adequate to accomplish this is defined as “therapeutically effective amount”. Effective amounts for each purpose will depend on the severity of the disease or injury as well as the weight and general state of the subject. It will be understood that determining an appropriate dosage may be achieved using routine experimentation, by constructing a matrix of values and testing different points in the matrix, which is all within the ordinary skills of a trained physician or veterinary.

The term “treatment” and “treating” as used herein means the management and care of a patient for the purpose of combating a condition, such as a disease or a disorder. The term is intended to include the full spectrum of treatments for a given condition from which the patient is suffering, such as administration of the active compound to alleviate the symptoms or complications, to delay the progression of the disease, disorder or condition, to alleviate or relief the symptoms and complications, and/or to cure or eliminate the disease, disorder or condition as well as to prevent the condition, wherein prevention is to be understood as the management and care of a patient for the purpose of combating the disease, condition, or disorder and includes the administration of the active compounds to prevent the onset of the symptoms or complications. The patient to be treated is preferably a mammal, in particular a human being, but it may also include animals, such as dogs, cats, cows, sheep, snakes and pigs.

In the present context, “increase” or “increasing” or similar, “preservation” or “preserving” or similar and “reduction of rate of loss” or similar of a given parameter, such as e.g. insulin secretion, β-cell function or number and/or size of β-cells, is to be understood in relation to an experiment comparing said parameter in the absence (control) and presence of the methods, compositions, kits or uses of the present invention. There is an increase in a given parameter, if the parameter is increased in the presence relative to the absence of the methods, compositions, kits or uses of the present invention. In an embodiment, said increase is above 5%, such as above 10% such as above 20% such as above 50% such as above 100%, wherein said increase in calculated relative to the value of the parameter in the absence of the methods, compositions, kits or uses of the present invention. The is a preservation in a given parameter, if the parameter is maintained at the initial level in the presence of the methods, compositions, kits or uses of the present invention while being reduced in their absence. There is a reduction of rate of loss of a given parameter, if the parameter is being reduced at a slower rate in the presence of the methods, compositions, kits or uses of the present invention than in their absence. In an embodiment, the rate of loss in the presence of the methods, compositions, kits or uses of the present invention is less than 95%, such as less than 90%, such as less than 70%, such as less than 50%, such as less than 20% of the rate of loss in the absence of the methods, compositions, kits or uses of the present invention.

### DESCRIPTION OF THE INVENTION

The present invention provides methods, uses, compositions and kits related to A GLP-1 receptor agonist and/or a DPP-IV inhibitor, in combination with a proton pump inhibitor and optionally a PPAR agonist.

The embodiments of the invention thus includes a GLP-1 receptor agonist in combination with a proton pump inhibitor. It also includes a DPP-IV inhibitor in combination with proton pump inhibitor. It also includes a A GLP-1 receptor agonist and a DPP-IV inhibitor, in combination with a proton pump inhibitor. The embodiments may also be combined with a PPAR agonist.

In an embodiment, the invention provides methods, uses, compositions and kits related to A GLP-1 receptor agonist and/or a DPP-IV inhibitor, in combination with a proton pump inhibitor and a PPAR agonist.

In an embodiment, the GLP-1 receptor agonist is a GLP-1 compound. Particular examples include GLP-1, such as human GLP-1 and exendin-4.

In an embodiment, the GLP-1 compound is an insulinotropic fragment of human GLP-1(1-37) or exendine-4(1-39), such as human GLP-1(7-37) wherein the amino acid residues in positions 1-6 of human GLP-1(1-37) have been deleted, and human GLP-1(7-36) where the amino acid residues in position 1-6 and 37 of human GLP-1(1-37) have been deleted, exendin-4(1-38) where amino acid residue 39 has been deleted from exendine-1(1-39) and exendin-4(1-31), where amino acid residue 32-39 have been deleted from exendine-4(1-39).

In an embodiment, the GLP-1 compound is an insulinotropic analogue of human GLP-1(1-37) or exendine-4(1-39), such as Met8-GLP-1(7-37) wherein the alanine in position 8 has been replaced by methionine and the amino acid residues in position 1 to 6 have been deleted relative to human GLP-1(1-37); Arg³⁴-GLP-1(7-37) wherein valine in position 34 has been replaced with arginine and the amino acid residues in position 1 to 6 have been deleted relative to human GLP-1(1-37); and Ser²Asp³-exendin-4(1-39) wherein the amino acid residues in position 2 and 3 have been replaced with serine and aspartic acid relative to exendine-4(1-39), respectively (this particular analogue also being known in the art as exendin-3).

In an embodiment, the GLP-1 compounds is an insulinotropic derivative of human GLP-1(1-37) or exendine-4(1-39), such as GLP-1(7-36)-amide, Arg³⁴, Lys²⁶(N^{ε}-(γ-Glu(N^{α}hexadecanoyl)))-GLP-1(7-37) and Tyr¹″-exendin-4(1-31)-amide. Particular mentioning is made of Arg³⁴, Lys²⁶(N^{ε}-(γ-Glu(N^{α}-hexadecanoyl)))-GLP-1(7-37).

In an embodiment, the proton pump inhibitor is selected from omeprazole, esomeprazole, lansoprazole, pantoprazole and rabeprazole, and in particular omeprazole or esomeprazole.

In an embodiment DPP-IV inhibitors are compounds such as vildagliptin, MK-0431, BMS-477118 (saxagliptin), PSN-9301, 823093, SYR-322, SYR-619, 815541, 825964, TA-6666 or TS-021;

In an embodiment, the PPAR agonist is a PPARα agonist. In particular, the PPARα agonist is a fibrate, such as clofibrate, bezafibrate, ciprofibrate, lofibrate, clofibride, gemfibrocil and fenofibrate. Particular mentioning is made of ciprofibrate.

In an embodiment, the PPAR agonist is a dual or triple acting agonist, such as MK-767, LY818, tesaglitazar, DRF-4158, LY465608, BMS-298585, netoglitazone and EML-16156.

In an embodiment, the methods of the present invention comprise the administration of therapeutically effective amounts of a GLP-1 receptor agonist in combination with a proton pump inhibitor and a PPAR agonist to a patient in need thereof.

In an embodiment, the methods of the present invention comprise the administration of therapeutically effective amounts of a GLP-1 compound in combination with a proton pump inhibitor and a PPARα agonist to a patient in need thereof.

In an embodiment, the invention relates to a method of delaying the progression of impaired glucose tolerance (IGT) to non-insulin dependent Type II diabetes mellitus, the method comprising administering to a patient in need thereof therapeutically effective amounts of a GLP-1 receptor agonist and/or a DPP-IV inhibitor, in combination with a proton pump inhibitor and optionally a PPAR agonist, such as a PPARα agonist.

In an embodiment, the invention relates to a method of delaying the progression of non-insulin dependent diabetes mellitus to insulin dependent Type II diabetes mellitus, the method comprising administering to a patient in need thereof therapeutically effective amounts of a GLP-1 receptor agonist and/or a DPP-IV inhibitor, in combination with a proton pump inhibitor and optionally a PPAR agonist, such as a PPARα agonist.

In an embodiment, the invention relates to a method of treating Type II diabetes mellitus, the method comprising administering to a patient in need thereof therapeutically effective amounts of a GLP-1 receptor agonist and/or a DPP-IV inhibitor, in combination with a proton pump inhibitor and optionally a PPAR agonist, such as a PPARα agonist.

In an embodiment, the invention relates to a method of treating Type I diabetes mellitus, the method comprising administering to a patient in need thereof therapeutically effective amounts of a GLP-1 receptor agonist and/or a DPP-IV inhibitor, in combination with a proton pump inhibitor and optionally a PPAR agonist, such as a PPARα agonist.

In an embodiment, the invention relates to a method of treating diseases according to the above, comprising administering to a patient in need thereof therapeutically effective amounts of a GLP-1 receptor agonist in combination with a proton pump inhibitor and optionally a PPAR agonist, such as a PPARα agonist.

In an embodiment, the invention relates to a method according to the above, comprising administering to a patient in need thereof therapeutically effective amounts of a DPP-IV inhibitor, in combination with a proton pump inhibitor and optionally a PPAR agonist, such as a PPARα agonist.

In an embodiment, the invention relates to the use of a GLP-1 receptor agonist and/or a DPP-IV inhibitor, in combination with a proton pump inhibitor and optionally a PPAR agonist, such as a PPARα agonist in the preparation of a medicament for delaying the progression of impaired glucose tolerance (IGT) to non-insulin dependent Type II diabetes mellitus

In an embodiment, the invention relates to the use a GLP-1 receptor agonist and/or a DPP-IV inhibitor, in combination with a proton pump inhibitor and optionally a PPAR agonist, such as a PPARα agonist in the preparation of a medicament for delaying the progression of non-insulin dependent diabetes mellitus to insulin dependent Type II diabetes mellitus.

In an embodiment, the invention relates to the use of a GLP-1 receptor agonist and/or a DPP-IV inhibitor, in combination with a proton pump inhibitor and optionally a PPAR agonist, such as a PPARα agonist in the manufacture of a medicament for treating Type II diabetes mellitus.

In an embodiment, the invention relates to the use of a GLP-1 receptor agonist and/or a DPP-IV inhibitor, in combination with a proton pump inhibitor and optionally a PPAR agonist, such as a PPARα agonist in the preparation of a medicament for treating Type I diabetes mellitus.

In an embodiment, the invention relates to the use according to the above of a GLP-1 receptor agonist in combination with a proton pump inhibitor and optionally a PPAR agonist, such as a PPARα agonist.

In an embodiment, the invention relates to the use of a DPP-IV inhibitor in combination with a proton pump inhibitor and optionally a PPAR agonist, such as a PPARα agonist in the preparation of a medicament for treating Type I diabetes mellitus.

As mentioned above, immunological break down of β-cells is part of the etiology of diabetes, and the methods, uses, compositions and kits of the present invention may thus advantageously comprise immunosuppressives and/or immunomodulators.

In an embodiment, immunosuppressives include rapamycin, corticosteroid, azathioprine, mycophenolate mofetil, everolimus, 6-mercaptopurine, alefacept, HLA-B2702 peptide, Azathioprine, Cladribine, cyclosporin A, dexamethasone, glatiramer acetate, gusperimus, infliximab, mycophenolate mofetil, muromonab-CD3, prednisolonecyclosporine, cyclophosphamide, methotrexate, mitoxantrone, demethimmunomycin, basiliximab, sirolimus, tacrolimus, antithymocyte immunoglobulin, efalizumab and daclizumab.

In an embodiment, immunomodulators include DiaPep277 and Diamyd.

In an embodiment, the invention relates to compositions comprising a GLP-1 receptor agonist and/or a DPP-IV inhibitor, a proton pump inhibitor, a PPAR agonist, such as a PPARα agonist, and optionally an immunosuppressive and/or an immunomodulator.

In an embodiment the compostions comprises A GLP-1 receptor agonist and a proton pump inhibitor. In embodiments it further comprises a PPAR agonist, such as a PPARα agonist, and optionally an immunosuppressive and/or an immunomodulator.

In an embodiment compositions comprises DPP-IV inhibitors and a proton pump inhibitor. In embodiments it further comprises a PPAR agonist, such as a PPARα agonist, and optionally an immunosuppressive and/or an immunomodulator.

In an embodiment, the present invention relates to a kit comprising several containers comprising the therapeutic agents to be used in the methods of the present invention, i.e. a container comprising a GLP-1 receptor agonist and/or a DPP-IV inhibitor, a container comprising a proton pump inhibitor, a container comprising a PPAR agonist, such as a PPARα agonist, and optionally a container comprising an immunosuppressive and/or immunomodulator. Depending on the whether or not the therapeutic agents can be formulated together, a container of the kit may comprise more than one of the active agents. In combination, the containers of the kit comprise all the active agents to be used in the methods of the present invention.

As described above, the methods of the present invention comprise the administration of a GLP-1 receptor agonist and/or a DPP-IV inhibitor, in combination with a proton pump inhibitor and optionally a PPAR agonist and/or an immunosuppressant and/or immunomodulator. These therapeutically active agents may be administered simultaneously sequentially, and with the same or different intervals, and it lies within the skills of a trained physician or veterinary to select a dosing regime which for a given patient exploits the present invention to its fullest.

In a particular embodiment, the patient is being administered a GLP-1 compound, regularly, such as one or more times a day or every second day, while the patient is only being administered a proton pump inhibitor, such as omeprazole, optionally in combination with a PPARα agonist, such as ciprofibrate, with much longer intervals, such as every 4, 5, 6 or 12 months.

In an embodiment, the invention relates to promotion of sales of the compositions and kits of the present invention, the promotion comprising the public distribution of information that the use of said compositions and kits is associated with β-cell proliferation or β-cell neogenesis. In an embodiment, said distribution of said information is achieved by a method selected from the group consisting of verbal communication, pamphlet distribution, print media, audio tapes, magnetic media, digital media, audiovisual media, billboards, advertising, newspapers, magazines, direct mailings, radio, television, electronic mail, braille, electronic media, banner ads, fiber optics, leaflets associated with packages comprising pharmaceutical compositions, and laser light shows.

### Pharmaceutical Compositions

The below description of pharmaceutical compositions is related to pharmaceutical compositions comprising all the therapeutically active agents to be used in the methods of the present invention. The description also relates to compositions comprising only one or more, but less than all of the therapeutically active agents to be used in the methods of the present invention. Two or more of such compositions may be presented as a kit to be used in the methods of the present invention, provided these compositions in combination comprise all the therapeutically active agents to be used in the methods of the present invention.

The compounds for methods according to the present invention may be administered alone or in combination with pharmaceutically acceptable carriers or excipients, in either single or multiple doses. The pharmaceutical compositions according to the invention may be formulated with pharmaceutically acceptable carriers or diluents as well as any other known adjuvants and excipients in accordance with conventional techniques such as those disclosed in Remington: The Science and Practice of Pharmacy, 20^{th} Edition, Gennaro, Ed., Mack Publishing Co., Easton, Pa., 2000.

The pharmaceutical compositions may be specifically formulated for administration by any suitable route such as the oral, rectal, nasal, pulmonary, topical (including buccal and sublingual), transdermal, intracisternal, intraperitoneal, vaginal and parenteral (including subcutaneous, intramuscular, intrathecal, intravenous and intradermal) route, the oral route being preferred. It will be appreciated that the preferred route will depend on the general condition and age of the subject to be treated, the nature of the condition to be treated and the active ingredient chosen. It will also be appreciated that if the therapeutically active agents to be used in the methods of the present invention is presented in more than one composition, i.e. presented as a kit, then each composition be administered by the same or different route.

Pharmaceutical compositions for oral administration include solid dosage forms such as hard or soft capsules, tablets, troches, dragees, pills, lozenges, powders and granules. Where appropriate, they can be prepared with coatings such as enteric coatings or they can be formulated so as to provide controlled release of the active ingredient such as sustained or prolonged release according to methods well known in the art.

Liquid dosage forms for oral administration include solutions, emulsions, aqueous or oily suspensions, syrups and elixirs.

Pharmaceutical compositions for parenteral administration include sterile aqueous and non-aqueous injectable solutions, dispersions, suspensions or emulsions as well as sterile powders to be reconstituted in sterile injectable solutions or dispersions prior to use. Depot injectable formulations are also contemplated as being within the scope of the present invention.

Other suitable administration forms include suppositories, sprays, ointments, cremes, gels, inhalants, dermal patches, implants etc.

A typical oral dosage for the proton pump inhibitor and the PPAR agonists is in the range of from about 1 to about 1000 mg/kg body weight per day, preferably from about 1 to about 500 mg/kg body weight per day, and more preferred from about 1 to about 100 mg/kg body weight per day administered in one or more dosages such as 1 to 3 dosages. A typical dose of a GLP-1 receptor agonist is in the range of about 0.1 ug/kg/day to about 40 ug/kg/day.

The exact dosage will depend upon the frequency and mode of administration, the sex, age, weight and general condition of the subject treated, the nature and severity of the condition treated and any concomitant diseases to be treated and other factors evident to those skilled in the art.

The formulations may conveniently be presented in unit dosage form by methods known to those skilled in the art. A typical unit dosage form for oral administration of the proton pump inhibitor or the PPAR agonists one or more times per day such as 1 to 3 times per day may contain from 0.05 to about 1000 mg, preferably from about 0.1 to about 500 mg, and more preferred from about 0.5 mg to about 200 mg. A typical formulation of a GLP-1 receptor agonist may contain from about 0.1 mg/ml to about 80 mg/ml.

For parenteral routes such as intravenous, intrathecal, intramuscular and similar administration, typically doses are in the order of about half the dose employed for oral administration.

For parenteral administration, solutions of the compounds for use according to the pre-sent invention in sterile aqueous solution, aqueous propylene glycol or sesame or peanut oil may be employed. Such aqueous solutions should be suitably buffered if necessary and the liquid diluent first rendered isotonic with sufficient saline or glucose. The aqueous solutions are particularly suitable for intravenous, intramuscular, subcutaneous and intraperitoneal administration. The sterile aqueous media employed are all readily available by standard techniques known to those skilled in the art.

Suitable pharmaceutical carriers include inert solid diluents or fillers, sterile aqueous solution and various organic solvents. Examples of solid carriers are lactose, terra alba, sucrose, cyclodextrin, talc, gelatine, agar, pectin, acacia, magnesium stearate, stearic acid and lower alkyl ethers of cellulose. Examples of liquid carriers are syrup, peanut oil, olive oil, phospholipids, fatty acids, fatty acid amines, polyoxyethylene and water. Similarly, the carrier or diluent may include any sustained release material known in the art, such as glyceryl monostearate or glyceryl distearate, alone or mixed with a wax. The pharmaceutical compositions formed by combining the therapeutically active agents to be used in the methods of the present invention and the pharmaceutically acceptable carriers are then readily administered in a variety of dosage forms suitable for the disclosed routes of administration. The formulations may conveniently be presented in unit dosage form by methods known in the art of pharmacy.

Formulations of the present invention suitable for oral administration may be presented as discrete units such as capsules or tablets, each containing a predetermined amount of the active ingredient, and which may include a suitable excipient. Furthermore, the orally available formulations may be in the form of a powder or granules, a solution or suspension in an aqueous or non-aqueous liquid, or an oil-in-water or water-in-oil liquid emulsion.

Compositions intended for oral use may be prepared according to any known method, and such compositions may contain one or more agents selected from the group consisting of sweetening agents, flavouring agents, colouring agents, and preserving agents in order to provide pharmaceutically elegant and palatable preparations. Tablets may contain the active ingredient in admixture with non-toxic pharmaceutically-acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example corn starch or alginic acid; binding agents, for example, starch, gelatine or acacia; and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in U.S. Pat. Nos. 4,356,108; 4,166,452; and 4,265,874, incorporated herein by reference, to form osmotic therapeutic tablets for controlled release.

Formulations for oral use may also be presented as hard gelatine capsules where the active ingredient is mixed with an inert solid diluent, for example, calcium carbonate, calcium phosphate or kaolin, or a soft gelatine capsules wherein the active ingredient is mixed with water or an oil medium, for example peanut oil, liquid paraffin, or olive oil.

Aqueous suspensions may contain the compound for use according to the present invention in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents may be a naturally-occurring phosphatide such as lecithin, or condensation products of an alkylene oxide with fatty acids, for example polyoxyethylene stearate, or condensation products of ethylene oxide with long chain aliphatic alcohols, for example, heptadecaethyl-eneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more colouring agents, one or more flavouring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as a liquid paraffin. The oily suspensions may contain a thickening agent, for example beeswax, hard paraffin or cetyl alcohol. Sweetening agents such as those set forth above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water provide the active compound in admixture with a dispersing or wetting agent, suspending agent and one or more preservatives. Suitable dispersing or wetting agents and suspending agents are exemplified by those already mentioned above. Additional excipients, for example, sweetening, flavouring, and colouring agents may also be present.

The pharmaceutical compositions comprising the therapeutically active agents to be used in the methods of the present invention may also be in the form of oil-in-water emulsions. The oily phase may be a vegetable oil, for example, olive oil or arachis oil, or a mineral oil, for example a liquid paraffin, or a mixture thereof. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of said partial esters with ethylene oxide, for example polyoxyethylene sorbitan monooleate. The emulsions may also contain sweetening and flavouring agents.

Syrups and elixirs may be formulated with sweetening agents, for example glycerol, propylene glycol, sorbitol or sucrose. Such formulations may also contain a demulcent, a preservative and flavouring and colouring agents. The pharmaceutical compositions may be in the form of a sterile injectable aqueous or oleaginous suspension. This suspension may be formulated according to the known methods using suitable dispersing or wetting agents and suspending agents described above. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution, and isotonic sodium chloride solution. In addition, sterile, fixed oils are conveniently employed as solvent or suspending medium. For this purpose, any bland fixed oil may be employed using synthetic mono- or diglycerides. In addition, fatty acids such as oleic acid find use in the preparation of injectables.

The compositions may also be in the form of suppositories for rectal administration of the compounds of the invention. These compositions can be prepared by mixing the drug with a suitable non-irritating excipient which is solid at ordinary temperatures but liquid at the rectal temperature and will thus melt in the rectum to release the drug. Such materials include cocoa butter and polyethylene glycols, for example.

For topical use, creams, ointments, jellies, solutions of suspensions, etc., containing the therapeutically active agents to be used in the methods of the invention are contemplated. For the purpose of this application, topical applications shall include mouth washes and gargles.

The compounds of the present invention may also be administered in the form of liposome delivery systems, such as small unilamellar vesicles, large unilamellar vesicles, and multilamellar vesicles. Liposomes may be formed from a variety of phospholipids, such as cholesterol, stearylamine, or phosphatidylcholines.

If a solid carrier is used for oral administration, the preparation may be tabletted, placed in a hard gelatine capsule in powder or pellet form or it can be in the form of a troche or lozenge. The amount of solid carrier will vary widely but will usually be from about 25 mg to about 1 g. If a liquid carrier is used, the preparation may be in the form of a syrup, emulsion, soft gelatine capsule or sterile injectable liquid such as an aqueous or non-aqueous liquid suspension or solution.

Pharmaceutical Protein Formulations

Another object of the present invention is to provide a pharmaceutical formulation comprising a compound which is present in a concentration from 0.001 mg/ml to 100 mg/ml, and wherein said formulation has a pH from 2.0 to 10.0. The formulation may further comprise a buffer system, preservative(s), tonicity agent(s), chelating agent(s), stabilizers and surfactants. In one embodiment of the invention the pharmaceutical formulation is an aqueous formulation, i.e. formulation comprising water. Such formulation is typically a solution or a suspension. In a further embodiment of the invention the pharmaceutical formulation is an aqueous solution. The term “aqueous formulation” is defined as a formulation comprising at least 50% w/w water. Likewise, the term “aqueous solution” is defined as a solution comprising at least 50% w/w water, and the term “aqueous suspension” is defined as a suspension comprising at least 50% w/w water.

In another embodiment the pharmaceutical formulation is a freeze-dried formulation, whereto the physician or the patient adds solvents and/or diluents prior to use.

In another embodiment the pharmaceutical formulation is a dried formulation (e.g. freeze-dried or spray-dried) ready for use without any prior dissolution.

In a further aspect the invention relates to a pharmaceutical formulation comprising an aqueous solution of a compound, and a buffer, wherein said compound is present in a concentration from 0.001 mg/ml or above, and wherein said formulation has a pH from about 2.0 to about 10.0.

In a another embodiment of the invention the pH of the formulation is selected from the list consisting of 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, and 10.0.

In a further embodiment of the invention the buffer is selected from the group consisting of sodium acetate, sodium carbonate, citrate, glycylglycine, histidine, glycine, lysine, arginine, sodium dihydrogen phosphate, disodium hydrogen phosphate, sodium phosphate, and tris(hydroxymethyl)-aminomethan, bicine, tricine, malic acid, succinate, maleic acid, fumaric acid, tartaric acid, aspartic acid or mixtures thereof. Each one of these specific buffers constitutes an alternative embodiment of the invention.

In a further embodiment of the invention the formulation further comprises a pharmaceutically acceptable preservative. In a further embodiment of the invention the preservative is selected from the group consisting of phenol, o-cresol, m-cresol, p-cresol, methyl p-hydroxybenzoate, propyl p-hydroxybenzoate, 2-phenoxyethanol, butyl p-hydroxybenzoate, 2-phenylethanol, benzyl alcohol, chlorobutanol, and thiomerosal, bronopol, benzoic acid, imidurea, chlorohexidine, sodium dehydroacetate, chlorocresol, ethyl p-hydroxybenzoate, benzethonium chloride, chlorphenesine (3p-chlorphenoxypropane-1,2-diol) or mixtures thereof. In a further embodiment of the invention the preservative is present in a concentration from 0.1 mg/ml to 20 mg/ml. In a further embodiment of the invention the preservative is present in a concentration from 0.1 mg/ml to 5 mg/ml. In a further embodiment of the invention the preservative is pre-sent in a concentration from 5 mg/ml to 10 mg/ml. In a further embodiment of the invention the preservative is present in a concentration from 10 mg/ml to 20 mg/ml. Each one of these specific preservatives constitutes an alternative embodiment of the invention. The use of a preservative in pharmaceutical compositions is well-known to the skilled person. For convenience reference is made to Remington: The Science and Practice of Pharmacy, 19^{th} edition, 1995.

In a further embodiment of the invention the formulation further comprises an isotonic agent. In a further embodiment of the invention the isotonic agent is selected from the group consisting of a salt (e.g. sodium chloride), a sugar or sugar alcohol, an amino acid (e.g. L-glycine, L-histidine, arginine, lysine, isoleucine, aspartic acid, tryptophan, threonine),

an alditol (e.g. glycerol (glycerine), 1,2-propanediol (propyleneglycol), 1,3-propanediol, 1,3-butanediol) polyethyleneglycol (e.g. PEG400), or mixtures thereof. Any sugar such as mono-, di-, or polysaccharides, or water-soluble glucans, including for example fructose, glucose, mannose, sorbose, xylose, maltose, lactose, sucrose, trehalose, dextran, pullulan, dextrin, cyclodextrin, soluble starch, hydroxyethyl starch and carboxymethylcellulose-Na may be used. In one embodiment the sugar additive is sucrose. Sugar alcohol is defined as a C4-C8 hydrocarbon having at least one —OH group and includes, for example, mannitol, sorbitol, inositol, galactitol, dulcitol, xylitol, and arabitol. In one embodiment the sugar alcohol additive is mannitol. The sugars or sugar alcohols mentioned above may be used individually or in combination. There is no fixed limit to the amount used, as long as the sugar or sugar alcohol is soluble in the liquid preparation and does not adversely effect the stabilizing effects achieved using the methods of the invention. In one embodiment, the sugar or sugar alcohol concentration is between about 1 mg/ml and about 150 mg/ml. In a further embodiment of the invention the isotonic agent is present in a concentration from 1 mg/ml to 50 mg/ml. In a further embodiment of the invention the isotonic agent is present in a concentration from 1 mg/ml to 7 mg/ml. In a further embodiment of the invention the isotonic agent is present in a concentration from 8 mg/ml to 24 mg/ml. In a further embodiment of the invention the isotonic agent is present in a concentration from 25 mg/ml to 50 mg/ml. Each one of these specific isotonic agents constitutes an alternative embodiment of the invention. The use of an isotonic agent in pharmaceutical compositions is well-known to the skilled person. For convenience reference is made to Remington: The Science and Practice of Pharmacy, 19^{th} edition, 1995.

In a further embodiment of the invention the formulation further comprises a chelating agent. In a further embodiment of the invention the chelating agent is selected from salts of ethylenediaminetetraacetic acid (EDTA), citric acid, and aspartic acid, and mixtures thereof. In a further embodiment of the invention the chelating agent is present in a concentration from 0.1 mg/ml to 5 mg/ml. In a further embodiment of the invention the chelating agent is present in a concentration from 0.1 mg/ml to 2 mg/ml. In a further embodiment of the invention the chelating agent is present in a concentration from 2 mg/ml to 5 mg/ml. Each one of these specific chelating agents constitutes an alternative embodiment of the invention. The use of a chelating agent in pharmaceutical compositions is well-known to the skilled person. For convenience reference is made to Remington: The Science and Practice of Pharmacy, 19^{th} edition, 1995.

In a further embodiment of the invention the formulation further comprises a stabilizer. The use of a stabilizer in pharmaceutical compositions is well-known to the skilled person. For convenience reference is made to Remington: The Science and Practice of Pharmacy, 19^{th} edition, 1995.

More particularly, compositions of the invention are stabilized liquid pharmaceutical compositions whose therapeutically active components include a polypeptide that possibly exhibits aggregate formation during storage in liquid pharmaceutical formulations. By “aggregate formation” is intended a physical interaction between the polypeptide molecules that results in formation of oligomers, which may remain soluble, or large visible aggregates that precipitate from the solution. By “during storage” is intended a liquid pharmaceutical composition or formulation once prepared, is not immediately administered to a subject. Rather, following preparation, it is packaged for storage, either in a liquid form, in a frozen state, or in a dried form for later reconstitution into a liquid form or other form suitable for administration to a subject. By “dried form” is intended the liquid pharmaceutical composition or formulation is dried either by freeze drying (i.e., lyophilization; see, for example, Williams and Polli (1984) J. Parenteral Sci. Technol. 38:48-59), spray drying (see Masters (1991) in Spray-Drying Handbook (5th ed; Longman Scientific and Technical, Essez, U.K.), pp. 491-676; Broadhead et al. (1992) Drug Devel. Ind. Pharm. 18:1169-1206; and Mumenthaler et al. (1994) Pharm. Res. 11:12-20), or air drying (Carpenter and Crowe (1988) Cryobiology 25:459-470; and Roser (1991) Biopharm. 4:47-53). Aggregate formation by a polypeptide during storage of a liquid pharmaceutical composition can adversely affect biological activity of that polypeptide, resulting in loss of therapeutic efficacy of the pharmaceutical composition. Furthermore, aggregate formation may cause other problems such as blockage of tubing, membranes, or pumps when the polypeptide-containing pharmaceutical composition is administered using an infusion system.

The pharmaceutical compositions of the invention may further comprise an amount of an amino acid base sufficient to decrease aggregate formation by the polypeptide during storage of the composition. By “amino acid base” is intended an amino acid or a combination of amino acids, where any given amino acid is present either in its free base form or in its salt form. Where a combination of amino acids is used, all of the amino acids may be present in their free base forms, all may be present in their salt forms, or some may be present in their free base forms while others are present in their salt forms. In one embodiment, amino acids to use in preparing the compositions of the invention are those carrying a charged side chain, such as arginine, lysine, aspartic acid, and glutamic acid. Any stereoisomer (i.e., L, D, or a mixture thereof) of a particular amino acid (e.g. methionine, histidine, imidazole, arginine, lysine, isoleucine, aspartic acid, tryptophan, threonine and mixtures thereof) or combinations of these stereoisomers, may be present in the pharmaceutical compositions of the invention so long as the particular amino acid is present either in its free base form or its salt form. In one embodiment the L-stereoisomer is used. Compositions of the invention may also be formulated with analogues of these amino acids. By “amino acid analogue” is intended a derivative of the naturally occurring amino acid that brings about the desired effect of decreasing aggregate formation by the polypeptide during storage of the liquid pharmaceutical compositions of the invention. Suitable arginine analogues include, for example, aminoguanidine, ornithine and N-monoethyl L-arginine, suitable methionine analogues include ethionine and buthionine and suitable cysteine analogues include S-methyl-L cysteine. As with the other amino acids, the amino acid analogues are incorporated into the compositions in either their free base form or their salt form. In a further embodiment of the invention the amino acids or amino acid analogues are used in a concentration, which is sufficient to prevent or delay aggregation of the protein.

In a further embodiment of the invention methionine (or other sulphuric amino acids or amino acid analogous) may be added to inhibit oxidation of methionine residues to methionine sulfoxide when the polypeptide acting as the therapeutic agent is a polypeptide comprising at least one methionine residue susceptible to such oxidation. By “inhibit” is intended minimal accumulation of methionine oxidized species over time. Inhibiting methionine oxidation results in greater retention of the polypeptide in its proper molecular form. Any stereoisomer of methionine (L or D) or combinations thereof can be used. The amount to be added should be an amount sufficient to inhibit oxidation of the methionine residues such that the amount of methionine sulfoxide is acceptable to regulatory agencies. Typically, this means that the composition contains no more than about 10% to about 30% methionine sulfoxide. Generally, this can be achieved by adding methionine such that the ratio of methionine added to methionine residues ranges from about 1:1 to about 1000:1, such as 10:1 to about 100:1.

In a further embodiment of the invention the formulation further comprises a stabilizer selected from the group of high molecular weight polymers or low molecular compounds. In a further embodiment of the invention the stabilizer is selected from polyethylene glycol (e.g. PEG 3350), polyvinyl alcohol (PVA), polyvinylpyrrolidone, carboxy/hydroxycellulose or derivates thereof (e.g. HPC, HPC-SL, HPC-L and HPMC), cyclodextrins, sulphur-containing substances as monothioglycerol, thioglycolic acid and 2-methylthioethanol, and different salts (e.g. sodium chloride). Each one of these specific stabilizers constitutes an alternative embodiment of the invention.

The pharmaceutical compositions may also comprise additional stabilizing agents, which further enhance stability of a therapeutically active polypeptide therein. Stabilizing agents of particular interest to the present invention include, but are not limited to, methionine and EDTA, which protect the polypeptide against methionine oxidation, and a non-ionic surfactant, which protects the polypeptide against aggregation associated with freezethawing or mechanical shearing.

In a further embodiment of the invention the formulation further comprises a surfactant. In a further embodiment of the invention the surfactant is selected from a detergent, ethoxylated castor oil, polyglycolyzed glycerides, acetylated monoglycerides, sorbitan fatty acid esters, polyoxypropylene-polyoxyethylene block polymers (eg. poloxamers such as Pluronic® F68, poloxamer 188 and 407, Triton X-100), polyoxyethylene sorbitan fatty acid esters, polyoxyethylene and polyethylene derivatives such as alkylated and alkoxylated derivatives (tweens, e.g. Tween-20, Tween-40, Tween-80 and Brij-35), monoglycerides or ethoxylated derivatives thereof, diglycerides or polyoxyethylene derivatives thereof, alcohols, glycerol, lectins and phospholipids (eg. phosphatidyl serine, phosphatidyl choline, phosphatidyl ethanolamine, phosphatidyl inositol, diphosphatidyl glycerol and sphingomyelin), derivates of phospholipids (eg. dipalmitoyl phosphatidic acid) and lysophospholipids (eg. palmitoyl lysophosphatidyl-L-serine and 1-acyl-sn-glycero-3-phosphate esters of ethanolamine, choline, serine or threonine) and alkyl, alkoxyl (alkyl ester), alkoxy (alkyl ether)-derivatives of lysophosphatidyl and phosphatidylcholines, e.g. lauroyl and myristoyl derivatives of lysophosphatidylcholine, dipalmitoylphosphatidylcholine, and modifications of the polar head group, that is cholines, ethanolamines, phosphatidic acid, serines, threonines, glycerol, inositol, and the positively charged DODAC, DOTMA, DCP, BISHOP, lysophosphatidylserine and lysophosphatidylthreonine, and glycerophospholipids (eg. cephalins), glyceroglycolipids (eg. galactopyransoide), sphingoglycolipids (eg. ceramides, gangliosides), dodecylphosphocholine, hen egg lysolecithin, fusidic acid derivatives—(e.g. sodium tauro-dihydrofusidate etc.), long-chain fatty acids and salts thereof C6-C12 (eg. oleic acid and caprylic acid), acylcarnitines and derivatives, N^{α}-acylated derivatives of lysine, arginine or histidine, or side-chain acylated derivatives of lysine or arginine, N^{α}-acylated derivatives of dipeptides comprising any combination of lysine, arginine or histidine and a neutral or acidic amino acid, N^{α}-acylated derivative of a tripeptide comprising any combination of a neutral amino acid and two charged amino acids, DSS (docusate sodium, CAS registry no [577-11-7]), docusate calcium, CAS registry no [128-49-4]), docusate potassium, CAS registry no [749]-09-0]), SDS (sodium dodecyl sulphate or sodium lauryl sulphate), sodium caprylate, cholic acid or derivatives thereof, bile acids and salts thereof and glycine or taurine conjugates, ursodeoxycholic acid, sodium cholate, sodium deoxycholate, sodium taurocholate, sodium glycocholate, N-Hexadecyl-N,N-dimethyl-3-ammonio-1-propanesulfonate, anionic (alkyl-aryl-sulphonates) monovalent surfactants, zwitterionic surfactants (e.g. N-alkyl-N,N-dimethylammonio-1-propanesulfonates, 3-cholamido-1-propyldimethylammonio-1-propanesulfonate, cationic surfactants (quaternary ammonium bases) (e.g. cetyltrimethylammonium bromide, cetylpyridinium chloride), non-ionic surfactants (eg. Dodecyl 5-D-glucopyranoside), poloxamines (eg. Tetronic's), which are tetrafunctional block copolymers derived from sequential addition of propylene oxide and ethylene oxide to ethylenediamine, or the surfactant may be selected from the group of imidazoline derivatives, or mixtures thereof. Each one of these specific surfactants constitutes an alternative embodiment of the invention.

The use of a surfactant in pharmaceutical compositions is well-known to the skilled person. For convenience reference is made to Remington: The Science and Practice of Pharmacy, 19^{th} edition, 1995.

In a further embodiment of the invention the formulation further comprises protease inhibitors such as EDTA (ethylenediamine tetraacetic acid) and benzamidineHCl, but other commercially available protease inhibitors may also be used. The use of a protease inhibitor is particular useful in pharmaceutical compositions comprising zymogens of proteases in order to inhibit autocatalysis.

It is possible that other ingredients may be present in the peptide pharmaceutical formulation of the present invention. Such additional ingredients may include wetting agents, emulsifiers, antioxidants, bulking agents, tonicity modifiers, chelating agents, metal ions, oleaginous vehicles, proteins (e.g., human serum albumin, gelatine or proteins) and a zwitterion (e.g., an amino acid such as betaine, taurine, arginine, glycine, lysine and histidine). Such additional ingredients, of course, should not adversely affect the overall stability of the pharmaceutical formulation of the present invention.

Pharmaceutical compositions containing a compound according to the present invention may be administered to a patient in need of such treatment at several sites, for example, at topical sites, for example, skin and mucosal sites, at sites which bypass absorption, for example, administration in an artery, in a vein, in the heart, and at sites which involve absorption, for example, administration in the skin, under the skin, in a muscle or in the abdomen.

Administration of pharmaceutical compositions according to the invention may be through several routes of administration, for example, lingual, sublingual, buccal, in the mouth, oral, in the stomach and intestine, nasal, pulmonary, for example, through the bronchioles and alveoli or a combination thereof, epidermal, dermal, transdermal, vaginal, rectal, ocular, for examples through the conjunctiva, uretal, and parenteral to patients in need of such a treatments.

Compositions of the current invention may be administered in several dosage forms, for example, as solutions, suspensions, emulsions, microemulsions, multiple emulsion, foams, salves, pastes, plasters, ointments, tablets, coated tablets, rinses, capsules, for example, hard gelatine capsules and soft gelatine capsules, suppositories, rectal capsules, drops, gels, sprays, powder, aerosols, inhalants, eye drops, ophthalmic ointments, ophthalmic rinses, vaginal pessaries, vaginal rings, vaginal ointments, injection solution, in situ transforming solutions, for example in situ gelling, in situ setting, in situ precipitating, in situ crystallization, infusion solution, and implants.

Compositions of the invention may further be compounded in, or attached to, for example through covalent, hydrophobic and electrostatic interactions, a drug carrier, drug delivery system and advanced drug delivery system in order to further enhance stability of the compound, increase bioavailability, increase solubility, decrease adverse effects, achieve chronotherapy well known to those skilled in the art, and increase patient compliance or any combination thereof. Examples of carriers, drug delivery systems and advanced drug delivery systems include, but are not limited to, polymers, for example cellulose and derivatives, polysaccharides, for example dextran and derivatives, starch and derivatives, poly(vinyl alcohol), acrylate and methacrylate polymers, polylactic and polyglycolic acid and block copolymers thereof, polyethylene glycols, carrier proteins, for example albumin, gels, for example, thermogelling systems, for example block co-polymeric systems well known to those skilled in the art, micelles, liposomes, microspheres, nanoparticulates, liquid crystals and dispersions thereof, L2 phase and dispersions there of, well known to those skilled in the art of phase behaviour in lipid-water systems, polymeric micelles, multiple emulsions, selfemulsifying, self-microemulsifying, cyclodextrins and derivatives thereof, and dendrimers.

Compositions of the current invention are useful in the formulation of solids, semisolids, powder and solutions for pulmonary administration, using, for example a metered dose inhaler, dry powder inhaler and a nebulizer, all being devices well known to those skilled in the art.

Compositions of the current invention are specifically useful in the formulation of controlled, sustained, protracting, retarded, and slow release drug delivery systems. More specifically, but not limited to, compositions are useful in formulation of parenteral controlled release and sustained release systems (both systems leading to a many-fold reduction in number of administrations), well known to those skilled in the art. Even more preferably, are controlled release and sustained release systems administered subcutaneous. Without limiting the scope of the invention, examples of useful controlled release system and compositions are hydrogels, oleaginous gels, liquid crystals, polymeric micelles, microspheres, nanoparticles,

Methods to produce controlled release systems useful for compositions of the current invention include, but are not limited to, crystallization, condensation, co-crystallization, precipitation, co-precipitation, emulsification, dispersion, high pressure homogenisation, encapsulation, spray drying, microencapsulating, coacervation, phase separation, solvent evaporation to produce microspheres, extrusion and supercritical fluid processes. General reference is made to Handbook of Pharmaceutical Controlled Release (Wise, D. L., ed. Marcel Dekker, New York, 2000) and Drug and the Pharmaceutical Sciences vol. 99: Protein Formulation and Delivery (MacNally, E. J., ed. Marcel Dekker, New York, 2000).

Parenteral administration may be performed by subcutaneous, intramuscular, intraperitoneal or intravenous injection by means of a syringe, optionally a pen-like syringe. Alternatively, parenteral administration can be performed by means of an infusion pump. A further option is a composition which may be a solution or suspension for the administration of the compound in the form of a nasal or pulmonal spray. As a still further option, the pharmaceutical compositions containing the [the protein] compound of the invention can also be adapted to transdermal administration, e.g. by needle-free injection or from a patch, optionally an iontophoretic patch, or transmucosal, e.g. buccal, administration.

The compound can be administered via the pulmonary route in a vehicle, as a solution, suspension or dry powder using any of known types of devices suitable for pulmonary drug delivery. Examples of these comprise of, but are not limited to, the three general types of aerosol-generating for pulmonary drug delivery, and may include jet or ultrasonic nebulizers, metered-dose inhalers, or dry powder inhalers (Cf. Yu J, Chien Y W. Pulmonary drug delivery: Physiologic and mechanistic aspects. Crit. Rev Ther Drug Carr Sys 14(4) (1997) 395-453).

Based on standardised testing methodology, the aerodynamic diameter (dₐ) of a particle is defined as the geometric equivalent diameter of a reference standard spherical particle of unit density (1 g/cm³). In the simplest case, for spherical particles, dₐ is related to a reference diameter (d) as a function of the square root of the density ratio as described by:

$${d}_{a} = \sqrt{\frac{ρ}{{ρ}_{a}}} d$$

Modifications to this relationship occur for non-spherical particles (cf. Edwards D A, Ben-Jebria A, Langer R. Recent advances in pulmonary drug delivery using large, porous inhaled particles. J Appl Physiol 84(2) (1998) 379-385). The terms “MMAD” and “MMEAD” are well-described and known to the art (cf. Edwards D A, Ben-Jebria A, Langer R and represents a measure of the median value of an aerodynamic particle size distribution. Recent advances in pulmonary drug delivery using large, porous inhaled particles. J Appl Physiol 84(2) (1998) 379-385). Mass median aerodynamic diameter (MMAD) and mass median effective aerodynamic diameter (MMEAD) are used inter-changeably, are statistical parameters, and empirically describe the size of aerosol particles in relation to their potential to deposit in the lungs, independent of actual shape, size, or density (cf. Edwards D A, Ben-Jebria A, Langer R. Recent advances in pulmonary drug delivery using large, porous inhaled particles. J Appl Physiol 84(2) (1998) 379-385). MMAD is normally calculated from the measurement made with impactors, an instrument that measures the particle inertial behaviour in air.

In a further embodiment, the formulation could be aerosolized by any known aerosolisation technology, such as nebulisation, to achieve a MMAD of aerosol particles less than 10 μm, more preferably between 1-5 μm, and most preferably between 1-3 μm. The preferred particle size is based on the most effective size for delivery of drug to the deep lung, where protein is optimally absorbed (cf. Edwards D A, Ben-Jebria A, Langer A, Recent advances in pulmonary drug delivery using large, porous inhaled particles. J Appl Physiol 84(2) (1998) 379-385).

Deep lung deposition of the pulmonal formulations comprising the compound may optional be further optimized by using modifications of the inhalation techniques, for example, but not limited to: slow inhalation flow (eg. 30 L/min), breath holding and timing of actuation.

The term “stabilized formulation” refers to a formulation with increased physical stability, increased chemical stability or increased physical and chemical stability.

The term “physical stability” of the protein formulation as used herein refers to the tendency of the protein to form biologically inactive and/or insoluble aggregates of the protein as a result of exposure of the protein to thermo-mechanical stresses and/or interaction with interfaces and surfaces that are destabilizing, such as hydrophobic surfaces and interfaces. Physical stability of the aqueous protein formulations is evaluated by means of visual inspection and/or turbidity measurements after exposing the formulation filled in suitable containers (e.g. cartridges or vials) to mechanical/physical stress (e.g. agitation) at different temperatures for various time periods. Visual inspection of the formulations is performed in a sharp focused light with a dark background. The turbidity of the formulation is characterized by a visual score ranking the degree of turbidity for instance on a scale from 0 to 3 (a formulation showing no turbidity corresponds to a visual score 0, and a formulation showing visual turbidity in daylight corresponds to visual score 3). A formulation is classified physical unstable with respect to protein aggregation, when it shows visual turbidity in daylight. Alternatively, the turbidity of the formulation can be evaluated by simple turbidity measurements well-known to the skilled person. Physical stability of the aqueous protein formulations can also be evaluated by using a spectroscopic agent or probe of the conformational status of the protein. The probe is preferably a small molecule that preferentially binds to a non-native conformer of the protein. One example of a small molecular spectroscopic probe of protein structure is Thioflavin T. Thioflavin T is a fluorescent dye that has been widely used for the detection of amyloid fibrils. In the presence of fibrils, and perhaps other protein configurations as well, Thioflavin T gives rise to a new excitation maximum at about 450 nm and enhanced emission at about 482 nm when bound to a fibril protein form. Unbound Thioflavin T is essentially non-fluorescent at the wavelengths.

Other small molecules can be used as probes of the changes in protein structure from native to non-native states. For instance the “hydrophobic patch” probes that bind preferentially to exposed hydrophobic patches of a protein. The hydrophobic patches are generally buried within the tertiary structure of a protein in its native state, but become exposed as a protein begins to unfold or denature. Examples of these small molecular, spectroscopic probes are aromatic, hydrophobic dyes, such as antrhacene, acridine, phenanthroline or the like. Other spectroscopic probes are metal-amino acid complexes, such as cobalt metal complexes of hydrophobic amino acids, such as phenylalanine, leucine, isoleucine, methionine, and valine, or the like.

The term “chemical stability” of the protein formulation as used herein refers to chemical covalent changes in the protein structure leading to formation of chemical degradation products with potential less biological potency and/or potential increased immunogenic properties compared to the native protein structure. Various chemical degradation products can be formed depending on the type and nature of the native protein and the environment to which the protein is exposed. Elimination of chemical degradation can most probably not be completely avoided and increasing amounts of chemical degradation products is often seen during storage and use of the protein formulation as well-known by the person skilled in the art. Most proteins are prone to deamidation, a process in which the side chain amide group in glutaminyl or asparaginyl residues is hydrolysed to form a free carboxylic acid. Other degradations pathways involves formation of high molecular weight transformation products where two or more protein molecules are covalently bound to each other through transamidation and/or disulfide interactions leading to formation of covalently bound dimer, oligomer and polymer degradation products (Stability of Protein Pharmaceuticals, Ahern. T. J. & Manning M. C., Plenum Press, New York 1992). Oxidation (of for instance methionine residues) can be mentioned, as another variant of chemical degradation. The chemical stability of the protein formulation can be evaluated by measuring the amount of the chemical degradation products at various time-points after exposure to different environmental conditions (the formation of degradation products can often be accelerated by for instance increasing temperature). The amount of each individual degradation product is often determined by separation of the degradation products depending on molecule size and/or charge using various chromatography techniques (e.g. SEC-HPLC and/or RP-HPLC).

Hence, as outlined above, a “stabilized formulation” refers to a formulation with increased physical stability, increased chemical stability or increased physical and chemical stability. In general, a formulation must be stable during use and storage (in compliance with recommended use and storage conditions) until the expiration date is reached.

In one embodiment of the invention the pharmaceutical formulation comprising the compound is stable for more than 6 weeks of usage and for more than 3 years of storage.

In another embodiment of the invention the pharmaceutical formulation comprising the compound is stable for more than 4 weeks of usage and for more than 3 years of storage.

In a further embodiment of the invention the pharmaceutical formulation comprising the compound is stable for more than 4 weeks of usage and for more than two years of storage.

In an even further embodiment of the invention the pharmaceutical formulation comprising the compound is stable for more than 2 weeks of usage and for more than two years of storage.

### Pharmacological Methods

β-cell function and number and/or size of β-cells may be measured as insulin and/or C-peptide secretion in response in vivo to a glucose load (OGTT, IVGTT), a mixed meal tolerance test (MMTT), Boost-test (carbohydrate enriched liquid meal) or a secretagogue, such as arginine, Katp channel blockers and incretin hormones, and particular mentioning is made of glucose load. Secreted insulin may be measured as serum insulin using enzyme-linked immunosorbent assay, DAKO insulin kit K6219, and C-peptide may be measured using radiolmmuno Assay, RIA using Novo antibody M1230, Diabetes Care 26: 832-36, 2003.

Combination of a GLP-1 Compound with a Proton Pump Inhibitor:

Diabetic Psammomys obesus were treated with vehicle, a GLP-1 compound alone (100 μg/kg, s.c.) or in combination with lanzoprazole (30 mg/kg, p.o.). At the end of the two week treatment period, the vehicle treated animals remained diabetic (BG 14.0±6.8 mM, HbA₁ 8.9±1.5%) whilst the animals in the GLP-1 compound alone groups had reduced levels of glycemia (BG 8.5±6.0 mM, HbA_{1C} 8.5±9.0%) and the animals treated with GLP-1 compound and lanzoprazole had become normoglycemic (morning BG 4.1±2.3 mM, HbA_{1C} 6.8±1.0% p<0.01 as compared to vehicle). There was no significant difference in body weight gain between the treatment groups.

1. A method of increasing insulin secretion, preserving insulin secretion or reducing the rate of loss of insulin secretion, the method comprising administering to a patient in need thereof therapeutically effective amounts of a GLP-1 receptor agonist and/or a DPP-IV inhibitor, in combination with a proton pump inhibitor.

2. A method of increasing β-cell function, preserving β-cell function or reducing the rate of loss of β-cell function in a patient, the method comprising administering to a patient in need thereof therapeutically effective amounts of a GLP-1 receptor agonist and/or a DPP-IV inhibitor, in combination with a proton pump inhibitor.

3. A method of increasing the number and/or size of β-cells, preserving the number and/or size of β-cells or reducing the rate of loss of the number and/or size of β-cells, the method comprising administering to a patient in need thereof therapeutically effective amounts of a GLP-1 receptor agonist and/or a DPP-IV inhibitor, in combination with a proton pump inhibitor.

4. A method of treating a disease benefiting from an increase in insulin secretion, a preservation of insulin secretion or a reduction in the rate of loss of insulin secretion, the method comprising administering to a patient in need thereof therapeutically effective amounts of a GLP-1 receptor agonist and/or a DPP-IV inhibitor, in combination with a proton pump inhibitor.

5. A method of treating a disease benefiting from an increase in β-cell function, a preservation of β-cell function or a reduction in the rate of loss of β-cell function, the method comprising administering to a patient in need thereof therapeutically effective amounts of a GLP-1 receptor agonist and/or a DPP-IV inhibitor, in combination with a proton pump inhibitor.

6. A method of treating a disease benefiting from an increase in the number and/or size of β-cells, a preservation in the number and/or size of β-cells or a reduction in the rate of loss in the number and/or size of β-cells, the method comprising administering to a patient in need thereof therapeutically effective amounts of a GLP-1 receptor agonist and/or a DPP-IV inhibitor, in combination with a proton pump inhibitor

7. A method of delaying the progression of impaired glucose tolerance (IGT) to non-insulin dependent Type II diabetes, the method comprising administering to a patient in need thereof therapeutically effective amounts of a GLP-1 receptor agonist and/or a DPP-IV inhibitor, in combination with a proton pump inhibitor.

8. A method of delaying the progression of non-insulin dependent Type II diabetes to insulin dependent Type II diabetes, the method comprising administering to a patient in need thereof therapeutically effective amounts of a GLP-1 receptor agonist and/or a DPP-IV inhibitor, in combination with a proton pump inhibitor.

9. A method of treating Type II diabetes, the method comprising administering to a patient in need thereof therapeutically effective amounts of a GLP-1 receptor agonist and/or a DPP-IV inhibitor, in combination with a proton pump inhibitor.

10. A method of treating Type I diabetes, the method comprising administering to a patient in need thereof therapeutically effective amounts of a GLP-1 receptor agonist and/or a DPP-IV inhibitor, in combination with a proton pump inhibitor.

11. The method according to claim 1, wherein the combination comprises a GLP-1 receptor agonist in combination with a proton pump inhibitor.

12. The method according to claim 11, wherein said GLP-1 receptor agonist is a GLP-1 compound.

13. The method according to claim 12, wherein said proton pump inhibitor is omeparzole or esomeprazole.

14. The method according to claim 13, which also comprises the administration of a PPAR agonist.

15. The method according to claim 14, wherein said PPAR agonist is a PPARα agonist.

16. The method according to claim 15, wherein said PPARα agonist is a fibrate.

17. The method according to claim 16, wherein said fibrate is ciprofibrate.

18. The method according to claim 1 which comprises the administration to said patient of an immunosuppressant and/or an immunomodulator.

19-33. (canceled)

34. A composition comprising GLP-1 receptor agonist and/or a DPP-IV inhibitor, and a proton pump inhibitor.

35. The composition according to claim 34, wherein said GLP-1 receptor agonist is a GLP-1 compound.

36. The composition according to claim 34, wherein said proton pump inhibitor is omeprazole or esomeprazole.

37. A composition according to claim 34, said composition further comprising a PPAR agonist.

38. The composition according to claim 37, wherein said PPAR agonist is a PPARα agonist.

39. The composition according to claim 38, wherein said PPARα agonist is a fibrate.

40. The composition according to claim 39, wherein said fibrate is ciprofibrate.

41. The composition according to claim 34, said composition further comprising an immunosuppressant and/or immunomodulator.

42. A kit comprising two or more containers (first containers), each container comprising at least one therapeutically active agent selected from a GLP-1 receptor agonist and/or a DPP-IV inhibitor, a proton pump inhibitor and/or a PPAR agonist, and wherein the containers together comprise all of said active compounds.

43. The kit according to claim 42, wherein said GLP-1 receptor agonist is a GLP-1 compound.

44. The kit according to claim 42 wherein said proton pump inhibitor is omeparzole or esomeprazole.

45. The kit according to claim 42, wherein said PPAR agonist is a PPARα agonist.

46. The kit according to claim 45 wherein said PPARα agonist is a fibrate.

47. The kit according to claim 46, wherein said fibrate is ciprofibrate.

48. The kit according to claim 42, said kit further comprising an immunosuppressant and/or immunomodulator, wherein said immunosuppressant/immunomodulator is comprised in said first containers or in a second container.

49. A method of promoting the sales of a composition or kit comprising a GLP-1 receptor agonist, a proton pump inhibitor, and optionally a PPAR agonist, said method comprising the public distribution of the information that administration of said composition or kit is associated with β-cell proliferation and/or β-cell neogenesis.
